(19) United States
(12) Patent Application Publication
(10) Pub. No.: US 2026/0048094 A1
(43) Pub. Date: Feb. 19, 2026

(54) COMPOSITION FOR PREVENTING, IMPROVING, OR MITIGATING KIDNEY-DEFICIENCY-ASSOCIATED SYMPTOMS OR CONDITIONS OF SKIN, HAIR, AND NAILS

(71) Applicant: SUMITOMO CHEMICAL COMPANY, LIMITED, Tokyo (JP)

(72) Inventors: Katsunori SASAKI, Tokyo (JP); Keiko HIRANO, Tokyo (JP); Takamitsu YOSHIDA, Nagoya-shi (JP)

(73) Assignee: SUMITOMO CHEMICAL COMPANY, LIMITED, Tokyo (JP)

(21) Appl. No.: 18/850,930

(22) PCT Filed: Feb. 22, 2023
(86) PCT No.: PCT/JP2023/006429

§ 371 Date: Sep. 25, 2024

(30) Foreign Application Priority Data
Apr. 1, 2022 (JP) 2022-061998

(51) Int. Cl. A61K 36/638; A61K 35/60; A61K 35/644; A61K 36/185; A61K 36/254; A61K 36/28; A61K 36/282; A61K 36/286; A61K 36/42; A61K 36/48; A61K 36/68; A61K 36/725; A61K 36/736; A61K 36/752; A61K 36/77; A61K 36/815; A61K 36/82; A61K 36/8945; A61K 36/8967; A61K 36/899; A61K 36/8994; A61K 36/9066; A61K 36/9068; A61K 47/36

(52) U.S. Cl. CPC A61K 36/638; A61K 35/60; A61K 35/644; A61K 36/185; A61K 36/254; A61K 36/28; A61K 36/282; A61K 36/286; A61K 36/42; A61K 36/48; A61K 36/577; A61K 36/68; A61K 36/725; A61K 36/736; A61K 36/752; A61K 36/77; A61K 36/815; A61K 36/82; A61K 36/8945; A61K 36/8967; A61K 36/899; A61K 36/8994; A61K 36/9066; A61K 36/9068; A61K 47/36; A61K 2236/33; A61K 2236/331; A61K 2236/51; A61K 2236/53

(57) ABSTRACT

A composition includes the following active components: component (1): at least one selected from the group consisting of Ligustrum fruit, yerbadetajo herb, Lilium bulb, Polygonatum Rhizome, Lycium fruit, Sesamum indicum, and black soybean; component (2): at least one selected from the group consisting of Suberect spatholobus stem, Longan aril, wild turmeric, zedoary root, Luffa vegetable sponge, Sparganium rhizome, safflower, Artemisia anomala, common fenugreek seed, Dipsacus japonicus root, Drynaria herb, sea horse, Cynomorium herb, and Astragalus Seed; component (3): at least one selected from the group consisting of tea leaf, plantago seed, plantago herb, coix seed, small red-bean, corn silk, Lophatherum herb, Benincasa seed, mallow seed, knotgrass, and Lygodium spore; and component (4): at least one selected from the group consisting of Dioscorea japonica Thunberg, Eleutherococcus senticosus Rhizome, honey, oriental bush cherry, ginger root, ripe tangerine peel, unripe tangerine peel, immature orange, malt starch syrup, jujube fruit, and non-glutinous rice.

### TECHNICAL FIELD

The present invention relates to a composition for preventing, improving, or mitigating kidney-deficiency-associated symptoms or conditions of skin, hair, and nails.

### BACKGROUND ART

In Chinese medicine that is a representative traditional medicine system and in Kampo medicine derived from the Chinese medicine, the theory of “life force, blood, and colorless bodily fluids” is systematized as a basic concept for interpreting the physical condition and constitution and the effects of the foods and pharmaceuticals ingested. This is a way of thinking that lists three elements of life force, blood, and colorless bodily fluids as the elements that make up the body, and classifies the physical condition and constitution as a result of imbalances such as lack of the elements, and stagnation of flow. The physical condition and constitution, and the effects of the foods, etc. ingested are interpreted based on the relationship between the “five organs” that conceptually classify the functions of the human body, that is, “liver/heart/spleen/lung/kidney” and the life force, blood, and colorless bodily fluids.

As described above, the five organs are “liver/heart/spleen/lung/kidney”, but do not correspond to anatomical organs, and are a conceptual classification of the functions of the human body. Among them, the “kidney” is considered to be the base of life, and has an essence storage function that stores energy indispensable for vital activities, that is, kidney essence. The stored kidney essence generates “life force”, and the action of the generated life force creates, replenishes, and stores the kidney essence. In this way, the kidney essence and the life force are repeatedly created, and are called vital spirit. Further, the kidney essence is a basic substance that produces “blood”, and if the kidney essence is enriched, the blood becomes abundant, and it is deemed that essence and blood share the same source (Non-Patent Document 1).

The state of a deficiency of kidney essence, that is, the kidney deficiency is understood as a lack of vital energy, and causes a disorder similar to the syndrome that appears due to so-called aging, and is associated with various malfunction symptoms accompanied with kidney qi deficiency that is a deficiency of vital spirit, and with blood deficiency that is a deficiency of blood generated by kidney essence (Non-Patent Document 1).

Examples of the malfunction symptoms associated with kidney deficiency include gray hair, hair loss, dryness/lack of firmness in hair, lack of hair gloss, hair waviness, dryness of skin, dryness of fingers and toes, loose facial skin due to dryness, fine wrinkles, itching of skin, lack of firmness and radiance on the face, mental instability, sleepiness, insomnia/sleep disorder, poor concentration due to insomnia, tinnitus, lightheadedness and dizziness/vertigo, vision impairment, muscle weakness, and heavy/sluggish in the body (Non-Patent Document 1).

Further, examples of the malfunction symptoms associated with kidney qi deficiency, which are caused by kidney deficiency, include chronic fatigue, shortness of breath, heavy/sluggish in the body, decrease in skin brightness due to aging, decrease in skin transparency due to aging, and weakness in the legs and lower back, and similarly, examples of the malfunction symptoms associated with blood deficiency, which are caused by kidney deficiency, include brittle/easily-broken nails, vertical lines on nails/poor nail color, numbness/weakness in hands and feet, and cold hands and feet (Non-Patent Document 1).

### PRIOR ART DOCUMENTS

### Non-Patent Document

Non-Patent Document 1: Nami Tatsumi, Practical Chinese Medicine: Basics and Applications in One Book, Gensosha (2009), p143-151, p168-173, p178-179

### SUMMARY OF THE INVENTION

### Problems to be Solved by the Invention

An object of the present invention is to provide a composition for preventing, improving, or mitigating kidney-deficiency-associated symptoms or conditions of skin, hair, and nails.

### Means for Solving the Problems

As a result of intensive studies to achieve the above object, the present inventors have found that it is possible to prevent, improve, or mitigate kidney-deficiency-associated symptoms or conditions of skin, hair, and nails by selecting and mixing groups of herbal medicines with specific medicinal benefit coverages and nature tastes. The present invention has been completed through further investigation based on this finding, and provides a novel composition including a herbal medicine component.

That is, the present invention is as follows.
[1]
A composition, including as active components the following components (1), (2), (3), and (4):
component (1): at least one kind of herbal medicine selected from the group consisting of Ligustrum fruit, yerbadetajo herb, Lilium bulb, Polygonatum Rhizome, Lycium fruit, Sesamum indicum, and black soybean;
component (2): at least one kind of herbal medicine selected from the group consisting of Suberect spatholobus stem, Longan aril, wild turmeric, zedoary root, Luffa vegetable sponge, Sparganium rhizome, safflower, Artemisia anomala, common fenugreek seed, Dipsacus japonicus root, Drynaria herb, sea horse, Cynomorium herb, and Astragalus Seed;
component (3): at least one kind of herbal medicine selected from the group consisting of tea leaf, plantago seed, plantago herb, coix seed, small red-bean, corn silk, Lophatherum herb, Benincasa seed, mallow seed, knotgrass, and Lygodium spore; and
component (4): at least one kind of herbal medicine selected from the group consisting of Dioscorea japonica Thunberg, Eleutherococcus senticosus Rhizome, honey, oriental bush cherry, ginger root, ripe tangerine peel, unripe tangerine peel, immature orange, malt starch syrup, jujube fruit, and non-glutinous rice.
[2]
The composition described in [1], in which the composition is for use in preventing, improving, or mitigating kidney-deficiency-associated symptoms or conditions of skin, hair, and nails.
[3]
The composition described in [2], in which the kidney-deficiency-associated symptoms or conditions of skin, hair, and nails is at least one selected from the group consisting of loose facial skin, fine wrinkles, gray hair, hair loss, dryness/lack of firmness in hair, brittle/easily-broken nails, vertical lines on nails/poor nail color, and dryness of skin.
[4]
The composition described in any one of [1] to [3], in which the composition is a food composition or a pharmaceutical composition.
[5]
A method for preventing, improving, or mitigating kidney-deficiency-associated symptoms or conditions of skin, hair, and nails, including administering the composition described in any one of [1] to [4] to a subject in need thereof.
[6]
The composition described in any one of [1] to [4], in which the composition is for use in preventing, improving, or mitigating kidney-deficiency-associated symptoms or conditions of skin, hair, and nails.
[7]
Use of the composition described in any one of [1] to [4], in producing a composition for preventing, improving, or mitigating kidney-deficiency-associated symptoms or conditions of skin, hair, and nails.
[8]
Use of the composition described in any one of [1] to [4], in preventing, improving, or mitigating kidney-deficiency-associated symptoms or conditions of skin, hair, and nails.

### Effect of the Invention

By mixing herbal medicines having specific medicinal benefit coverages and nature tastes, the composition of the present invention replenishes and circulates the blood that is the base for supplementing kidney essence, excretes excess water that has stagnated in the body, and further synergistically exerts an action of components that contribute to the excretion of toxins, as a result of which kidney-deficiency-associated symptoms or conditions of skin, hair, and nails can be prevented, improved, or mitigated. In particular, the composition is effective for the symptoms such as loose facial skin, fine wrinkles, gray hair, hair loss, dryness/lack of firmness in hair, brittle/easily-broken nails, vertical lines on nails/poor nail color, and dryness of skin.

Further, the composition of the present invention can widely prevent, improve, or mitigate the symptoms such as chronic fatigue/shortness of breath, heavy/sluggish in the body, and insomnia/sleep disorder as a health promotion effect associated with the elimination of kidney deficiency.

### MODE FOR CARRYING OUT THE INVENTION

The “kidney deficiency” refers to a state of deficiency of the kidney essence that is energy indispensable for vital activities, causes a disorder similar to the syndrome that appears due to aging, and further is associated with various malfunction symptoms accompanied with kidney qi deficiency that is a deficiency of vital spirit, and blood deficiency that is a deficiency of blood. As the causes, congenital weakness, malnutrition, sexual incontinence, exhaustion due to chronic diseases, and aging are known. Examples of the symptoms of kidney deficiency include gray hair, hair loss, dryness/lack of firmness in hair, lack of hair gloss, hair waviness, dryness of skin, dryness of fingers and toes, loose facial skin due to dryness, fine wrinkles, itching of skin, lack of firmness and radiance on the face, mental instability, sleepiness, insomnia/sleep disorder, poor concentration due to insomnia, tinnitus, lightheadedness and dizziness/vertigo, vision impairment, muscle weakness, and heavy/sluggish in the body. Further, examples of the malfunction symptoms associated with kidney qi deficiency, which are caused by kidney deficiency, include chronic fatigue, shortness of breath, heavy/sluggish in the body, decrease in skin brightness due to aging, decrease in skin transparency due to aging, and weakness in the legs and lower back, and similarly, examples of the malfunction symptoms associated with blood deficiency, which are caused by kidney deficiency, include brittle/easily-broken nails, vertical lines on nails/poor nail color, numbness/weakness in hands and feet, and cold hands and feet.

In the present specification, the kidney-deficiency-associated symptoms or conditions of skin, hair, and nails are mainly symptoms relating to kidney deficiency, and further refer to various symptoms brought about by the kidney qi deficiency/blood deficiency caused by kidney deficiency. More specific examples of the symptoms include at least one selected from the group consisting of loose facial skin, fine wrinkles, gray hair, hair loss, dryness/lack of firmness in hair, brittle/easily-broken nails, vertical lines on nails/poor nail color, and dryness of skin, and preferably include gray hair, hair loss, dryness/lack of firmness in hair, brittle/easily-broken nails, and dryness of skin.

The composition of the present embodiment includes components (1), (2), (3), and (4) as the active components. The components (1), (2), (3), and (4) each contain a group of herbal medicines having specific medicinal benefit coverages, and nature tastes. The medicinal benefit coverage, nature taste, and efficacy of each herbal medicine are as described later. In addition, for each herbal medicine, the generally known origin and site may be used, and the details are as described later.

In this regard, the medicinal benefit coverage is one of the concepts used in Chinese medicine (TCM), refers to which part of the human body a herbal medicine acts on, and indicates the action site where the medicinal effect appears. The medicinal benefit coverage is composed of so-called “five organs”, that is, “liver/heart/spleen/lung/kidney”, and the five organs in Chinese medicine do not correspond to anatomical organs, but rather mean “functions” of the human body. The liver is related to the autonomic nerve, the emotion, the motor nerve, the gynecological system, and the liver, stores the blood, and controls the blood so that the life force and blood smoothly flow. The heart is related to the brain and the circulatory system including the heart, and has a function of circulating the blood throughout the body and nourishing the body. The spleen is related to the digestive system, allergies, and part of water metabolism, digests and absorbs foods and drinks, and creates “life force, blood, and colorless bodily fluids”, which are the basics of the body. The lung is related to the respiratory system, the skin, part of water metabolism, and allergies, works together with the spleen to create “life force”, spreads the life force throughout the body, regulates the flow of colorless bodily fluids, protects the body from external bad vibes, and is closely related to the immune system. The kidney is related to the reproductive system, the urinary system, the endocrine system, and aging and development, stores the source of vital energy, controls the water metabolism of the whole body, and produces and reabsorbs urine. In Chinese medicine, the five organs work together to generate and store the vital spirit of the body, and control the mental activity. In addition, the medicinal benefit coverage may consist not only of the five organs but also of the “six hollow organs”, that is, “stomach/small intestine/large intestine/bladder/gallbladder/triple heater”.

The nature taste (also referred to as “qi taste”) is a type of classification system that indicates a nature of a herbal medicine, each taste is related to the five organs, and the taste itself has its own role. Most commonly, the nature taste indicates a nature of cooling and warming the human body by five natures of “cold/cool/flat/warm/heat”, indicates a taste at the time of ingestion by five tastes of “hot/sweet/acid/bitter/salty”, and is also an indicator of the efficacy. In the five tastes, “light/astringent” may also be added in some cases. The “heat” and “warm” have a function of warding off cold, warming the body, stimulating the body, and activating the body. In addition, the “cold” and “cool” have a function of cooling the body heat, detoxifying, and calming the mind, and the “flat” refers to a nature of neither warming nor cooling. The “cold” may be further divided into “great cold”, “cold”, and “slight cold” depending on the degree. The “hot” is related to the lung, and has an effect of sweating, circulating the life force, and diverting oneself. The “sweet” is related to the spleen, and has a supplementing or mitigating effect. The “acid” is related to the liver, and has a tightening effect. The “bitter” is related to the heart, and has an effect of cooling heat, and drying and lowering dampness. The “salty” is related to the kidney, and has an effect of softening hard things, and moistening and lowering them. The nature taste may be subdivided into “great cold”, “slight bitter” and the like depending on the degree.

The medicinal benefit coverage and nature taste are set for almost all the herbal medicines, herbal medicines that are coincident in the medicinal benefit coverage and/or the nature taste tend to have the same efficacy. The medicinal benefit coverage and nature taste are representatives of technical ideas that help to systematize the efficacy/prescription design of a huge number of herbal medicines.

### <Component (1)>

Component (1) is a herbal medicine having a medicinal benefit coverage of “kidney” or “heart”, and is specifically at least one kind selected from the group consisting of Ligustrum fruit, yerbadetajo herb, Lilium bulb, Polygonatum Rhizome, Lycium fruit, Sesamum indicum, and black soybean. Among these herbal medicines, from the viewpoint of the availability (supply stability), at least one kind selected from the group consisting of Ligustrum fruit, yerbadetajo herb, Lilium bulb, Lycium fruit, Sesamum indicum, and black soybean is preferable; from the viewpoint of exerting the effect with a small amount of raw material herbal medicine, at least one kind selected from the group consisting of Ligustrum fruit, yerbadetajo herb, Lilium bulb, Lycium fruit, and Sesamum indicum is more preferable; and from the viewpoint of the efficacy, Ligustrum fruit is particularly preferable. The component (1) may be one or more kinds of herbal medicines, two or more kinds of herbal medicines, three or more kinds of herbal medicines, or four or more kinds of herbal medicines.

Ligustrum fruit (Joteishi) is a dried ripe fruit made by drying as it is or after steaming or blanching a ripe fruit of Ligustrum lucidum (Ligustrum lucidum W. T. Aiton) or Japanese privet (Ligustrum japonicum Thunb.), the nature taste is cool, and sweet/bitter, the medicinal benefit coverage is liver/kidney, and the efficacy is nourishing the kidney nourishing the liver/black hair and improving the eyesight/clearing heat due to deficiency. The main components are oleanolic acid, ursolic acid, and mannitol.

Yerbadetajo herb (Kanrensou) is a dried aerial part of Takasaburou (Eclipta prostrata L.), the nature taste is cold, and sweet/acid, the medicinal benefit coverage is liver/kidney, and the efficacy is nourishing the liver and benefiting the kidney/black hair and consolidating the teeth, and cooling blood for hemostasis. The main components are saponin, tannin, and vitamin A.

Lilium bulb (Byakugou) is dried scale leaves made usually by steaming and drying scale leaves of tiger lily (Lilium lancifolium Thunb.), Lilium brownii F. E. Brown, Hakatayuri (Lilium brownii F. E. Brown var. colchesteri Wils.), or Itohayuri (Lilium pumilum DC.), the nature taste is slight cold, and sweet, the medicinal benefit coverage is heart/lung, and the efficacy is moistening the lung and relieving cough, and clearing the heart and soothing the nerves. The main components are starch, a protein, fat, and alkaloid.

Polygonatum Rhizome (Ousei) is a dried rhizome made usually by steaming and drying a rhizome of Polygonatum kingianum Collettet Hemsley, Kagikurumabanarukoyuri (Polygonatum sibiricum Red.), Polygonatum cyrtonema Hua, or narukoyuri (Polygonatum falcatum A. Gray), the nature taste is flat, and sweet, the medicinal benefit coverage is spleen/lung/kidney, and the efficacy is moistening the lung and relieving cough, and clearing the heart and soothing the nerves. The main components are mucilaginous polysaccharides, flavonoid, and alkaloid.

Lycium fruit (Kukoshi) is a dried fruit of Chinese boxthorn (Lycium chinense Mill.), or tea tree (Lycium barbarum L.), the nature taste is flat, and sweet, the medicinal benefit coverage is liver/kidney/lung, and the efficacy is nourishing the liver and kidney/improving eyesight, and moistening the lung. The main components are betaine, and zeaxanthin.

Sesamum indicum (Gomanin) is a dried ripe fruit of sesame seed (Sesamum indicum L.), the nature taste is flat, and sweet, the medicinal benefit coverage is spleen/lung/liver/kidney, and the efficacy is nourishing the liver and kidney/invigorating the essence and blood, and moistening dryness and smoothing the intestines. The main components are lignan, and a fatty acid.

Black soybean (Kokuzu) is a dried ripe fruit of soybean (Glycine max (L.) Merr. subsp. Max), the nature taste is flat, and sweet, the medicinal benefit coverage is liver/kidney, and the efficacy is nourishing yin and replenishing blood/diuresis, dispelling pathogenic wind and relieving convulsion, and removing toxicity. The main components are a protein, fat, isoflavones, and saponin.

### <Component (2)>

Component (2) is a herbal medicine having a medicinal benefit coverage of “liver” or “heart”, and is specifically at least one kind selected from the group consisting of Suberect spatholobus stem, Longan aril, wild turmeric, zedoary root, Luffa vegetable sponge, Sparganium rhizome, safflower, Artemisia anomala, common fenugreek seed, Dipsacus japonicus root, Drynaria herb, sea horse, Cynomorium herb, and Astragalus Seed. Among these herbal medicines, from the viewpoint of the availability (supply stability), at least one kind selected from the group consisting of Suberect spatholobus stem, Longan aril, wild turmeric, zedoary root, Luffa vegetable sponge, safflower, and common fenugreek seed is preferable; and from the viewpoint of the efficacy, at least one kind selected from the group consisting of Suberect spatholobus stem, wild turmeric, zedoary root, safflower, and common fenugreek seed is more preferable, and at least one kind selected from the group consisting of Suberect spatholobus stem, wild turmeric, zedoary root, and common fenugreek seed is particularly preferable. The component (2) may be one or more kinds of herbal medicines, two or more kinds of herbal medicines, three or more kinds of herbal medicines, or four or more kinds of herbal medicines.

Suberect spatholobus stem (Keikettou) is a dried climbing stem of Mitsukatou (Spatholobus suberectus Dunn), the nature taste is warm, and bitter/slight sweet, the medicinal benefit coverage is liver/kidney, and the efficacy is promoting blood circulation and replenishing blood, and relieving rigidity of muscle for removing obstruction in collaterals. The main components are a phenolic compound, iron, and an amino acid.

Longan aril (Ryuganniku) is a dried aril of longan (Euphoria longana Lam), the nature taste is flat, and sweet, the medicinal benefit coverage is heart/spleen, and the efficacy is nourishing the heart and blood, tranquillization, and invigorating spleen qi. The main components are saccharides, and organic acid.

Wild turmeric (Kyouou) is a dried rhizome made usually by blanching and drying a rhizome as it is or after removing the cork layer of turmeric (Curcuma longa L.), the nature taste is warm, and bitter/hot, the medicinal benefit coverage is spleen/liver, and the efficacy is breaking blood circulation and activating qi flowing/restoring menstrual flow and relieving pain, and dispelling pathogenic wind and overcoming dampness. The main components are essential oil, polyphenol, and starch.

Zedoary root (Gajutsu) is a dried rhizome made usually by blanching and drying a rhizome of zedoary (Curcuma zedoaria Roscoe, Curcuma phaeocaulis Valeton, Curcuma kwangsiensis S. G. Lee et C. F. Liang), the nature taste is warm, and bitter/hot, the medicinal benefit coverage is liver/spleen, and the efficacy is activating qi flowing and breaking blood, and eliminating accumulation and relieving pain. The main components are essential oil, polyphenol, and starch.

Luffa vegetable sponge (Shikaraku) is a dried vascular bundle of ripe fruit of loofah (Luffa cylindrica (L.) M. Roem.), the nature taste is flat, and sweet, the medicinal benefit coverage is lung/stomach/liver, and the efficacy is activating blood circulation for removing obstruction in collaterals. The main components are polysaccharides, and a phenolic compound.

Sparganium rhizome (Sanryou) is a dried tuber excluding the outer skin of bur reed (Sparganium stoloniferum (Graebn.) Buch.-Ham. ex Juz.), the nature taste is flat, and bitter, the medicinal benefit coverage is liver/spleen, and the efficacy is breaking blood and activating qi flowing, and eliminating accumulation and relieving pain. The main components are essential oil, and starch.

Safflower (Kouka) is a tubular flower as it is or after removing most of the yellow pigment, and sometimes in a plate shape made by pressing the flower of Carthamus tinctorius L., the nature taste is warm, and hot, the medicinal benefit coverage is heart/liver, and the efficacy is promoting blood circulation and restoring menstrual flow, and eliminating blood stasis and relieving pain. The main component is flavonoid.

Artemisia anomala (Ryukido) is a dried whole grass of Ritouzanyomogi (Artemisia anomala S. Moore), the nature taste is warm, and hot/bitter, the medicinal benefit coverage is heart/liver/spleen/lung/kidney/bladder, and the efficacy is breaking blood circulation and restoring menstrual flow/removing distension and relieving pain, hemostasis for removing edema, clearing heat and diuresis, and lowering qi and removing distension. The main component is essential oil.

Common fenugreek seed (Koroha) is a dried ripe seed of fenugreek (Trigonella foenum-graecum L.), the nature taste is great warm, and bitter, the medicinal benefit coverage is liver/kidney, and the efficacy is warming the kidney and yang-qi, expelling cold and dampness, and relieving pain. The main components are polysaccharides, saponin, flavonoid, and alkaloid.

Dipsacus japonicus root (Zokudan) is a dried root of Tounabena (Dipsacus asper Wall. ex Henry), the nature taste is slight warm, and bitter/sweet/hot, the medicinal benefit coverage is liver/kidney, and the efficacy is replenishing the liver and kidney, promoting system for relieving pain, stimulating blood vessels, healing the muscles and bones, and consolidating channel for arresting collapse/tranquilizing fetus to prevent miscarriage. The main components are alkaloid, and essential oil.

Drynaria herb (Kotsusaiho) is a dried rhizome made by drying a rhizome as it is or after removing the scales by burning of Hakamauraboshi (Drynaria fortunei (Kunze ex Mett.) J. Sm.), the nature taste is warm, and bitter, the medicinal benefit coverage is liver/kidney, and the efficacy is tonifying the kidney, and promoting blood circulation for healing the muscles. The main components are starch, saccharides, and flavonoid.

Sea horse (Kaiba) is a dried whole or a dried part made by drying after removing the film and internal organs from a whole of great seahorse (Hippocampus kelloggi Jordan et Snyder), Ibaratatsu (Hippocampus histrix Kaup), Hippocampus kuda Bleeker, Hippocampus trimaculatus Leach, or Sangotatsu (Hippocampus japonicus Kaup), the nature taste is warm, and sweet, the medicinal benefit coverage is liver/kidney, and the efficacy is tonifying the kidney and strengthening yang-qi, improving qi flowing and calming panting, arresting enuresis, and harmonizing qi flowing and promoting blood circulation. The main component is terpenoid.

Cynomorium herb (Sayou) is a dried fleshy stem of Oshagujitake (Cynomorium songaricum Rupr.), the nature taste is warm, and sweet, the medicinal benefit coverage is liver/kidney, and the efficacy is tonifying the kidney and yang-qi, benefiting the essence and blood, nourishing the muscles, and clearing the intestine for curing constipation. The main components are tannin, triterpene, anthocyanin, and an amino acid.

Astragalus Seed (Shaenshi) is a dried ripe seed of Tsurugenge (Astragalus complanatus R. Br.), the nature taste is warm, and sweet, the medicinal benefit coverage is liver/kidney, and the efficacy is tonifying the kidney and yang-qi, benefiting the essence, consolidating the essence, reducing urination, and nourishing the liver for improving eyesight. The main components are flavonoid, and alkaloid.

### <Component (3)>

Component (3) is a herbal medicine having a medicinal benefit coverage of “large intestine”, “small intestine”, or “stomach” and a nature of “cold”, “slight cold”, or “flat” in the five natures, and is specifically at least one kind selected from the group consisting of tea leaf, plantago seed, plantago herb, coix seed, small red-bean, corn silk, Lophatherum herb, Benincasa seed, mallow seed, knotgrass, and Lygodium spore. Among these herbal medicines, from the viewpoint of the availability (supply stability), at least one kind selected from the group consisting of tea leaf, plantago seed, plantago herb, coix seed, small red-bean, corn silk, and Lophatherum herb is preferable; and from the viewpoint of exerting the effect with a small amount of raw material herbal medicine, at least one kind selected from the group consisting of tea leaf, plantago seed, or plantago herb is more preferable; and from the viewpoint of the ease of processing for commercialization, at least one kind selected from the group consisting of tea leaf, and plantago herb is particularly preferable. The component (3) may be one or more kinds of herbal medicines, two or more kinds of herbal medicines, three or more kinds of herbal medicines, or four or more kinds of herbal medicines.

Tea leaf (Chayou) is a dried leaf (often with a branch tip) of tea (Camellia sinensis (L.) Kuntze), the nature taste is slight cold, and bitter/slight sweet, the medicinal benefit coverage is heart/lung/liver/kidney/spleen/stomach, and the efficacy is dispelling pathogenic wind/refreshing the head and eyes, clearing heat and lowering fire/resolving summer-heat, reducing fever and toxicity/relieving dysentery, and diuresis. The main components are alkaloid, flavonoid, and tannin.

Plantago seed (Shazenshi) is a dried ripe seed of plantain (Plantago asiatica L.), the nature taste is cold, and sweet/light, the medicinal benefit coverage is liver/kidney/lung/small intestine, and the efficacy is clearing heat and promoting urination, diffusing dampness and relieving diarrhea, removing liver-fire for improving eyesight, and dissipating phlegm and relieving cough. The main components are mucilaginous polysaccharides, and iridoid glucosides.

Plantago herb (Shazensou) is a dried whole grass in flowering time of plantain (Plantago asiatica L.), the nature taste is cold, and sweet/light, the medicinal benefit coverage is liver/kidney/lung/small intestine, and the efficacy is clearing heat and promoting urination, diffusing dampness and relieving diarrhea, removing liver-fire for improving eyesight, and dissipating phlegm and relieving cough. The main components are iridoid glucosides, and flavonoid.

Coix seed (Yokuinin) is a dried ripe seed excluding the seed coat of adlay (Coix lacryma-jobi var.ma-yuen (Rom. Caill.) Stapf), the nature taste is slight cold, and sweet/light, the medicinal benefit coverage is spleen/stomach/lung, and the efficacy is clearing and disinhibiting dampness-heat, removing dampness and relieving numbness, expelling pus and removing edema, and invigorating the spleen and relieving diarrhea. The main components are starch, and a protein.

Small red-bean (Sekishouzu) is a dried ripe seed of rice bean (Vigna umbellata (Thunb.) Ohwi et Ohashi), or adzuki bean (Vigna angularis (Willd.) Ohwi et Ohashi), the nature taste is slight cold, sweet/acid, the medicinal benefit coverage is heart/small intestine, and the efficacy is inducing diuresis and removing edema, clearing heat and diuresis, curing jaundice, and removing toxicity and expelling pus. The main components are a protein, isoflavones, saponin, and anthocyanin.

Corn silk (Gyokubeiju) is a dried style of maize (Zea mays L.), the nature taste is flat, and sweet, the medicinal benefit coverage is liver/kidney/bladder/heart/small intestine, and the efficacy is inducing diuresis and removing edema, and curing jaundice. The main components are saponin, and flavonoid.

Lophatherum herb (Tanchikuyou) is a dried stem and dried leaves of Sasakusa (Lophatherum gracile Brongn.), the nature taste is slight cold, and sweet/light, the medicinal benefit coverage is heart/lung/small intestine, and the efficacy is inducing diuresis for stranguria, and clearing the heart and eliminating agitation. The main components are triterpenoid, saccharides, and an amino acid.

Benincasa seed (Tougashi) is a dried ripe seed of wax gourd (Benincasa cerifera Savi) or Benincasa cerifera Savi forma emarginata K. Kimura et Sugiyama, the nature taste is slight cold, and sweet, the medicinal benefit coverage is lung/stomach/large intestine/small intestine, and the efficacy is clearing heat, and inducing diuresis and removing edema. The main components are a fatty acid, and alkaloid.

Mallow seed (Toukishi) is a dried ripe seed of Fuyuaoi (Malva verticillata L.), the nature taste is cold, and sweet, the medicinal benefit coverage is small intestine/large intestine/bladder, and the efficacy is inducing diuresis for stranguria, clearing the intestine for curing constipation, and promoting the flow of breast milk. The main components are fatty oil, and a protein.

Knotgrass (Henchiku) is a dried aerial part of prostrate knotweed (Polygonum aviculare L.), the nature taste is flat, and bitter, the medicinal benefit coverage is stomach/bladder, and the efficacy is promoting diuresis for stranguria, and killing insects and relieve itching. The main components are flavone glycosides, and tannin. Lygodium spore (Kakinsha) is dried ripe spores of Kanikusa (Lygodium japonicum (Thunb.) Sw.), the nature taste is cold, and sweet/salty, the medicinal benefit coverage is bladder/small intestine, and the efficacy is promoting diuresis for stranguria. The main components are fatty oil, and lygodine.

### <Component (4)>

Component (4) is a herbal medicine having a medicinal benefit coverage of “spleen” or “kidney”, and is specifically at least one kind selected from the group consisting of Dioscorea japonica Thunberg, Eleutherococcus senticosus Rhizome, honey, oriental bush cherry, ginger root, ripe tangerine peel, unripe tangerine peel, immature orange, malt starch syrup, jujube fruit, and non-glutinous rice. Among these herbal medicines, from the viewpoint of the availability (supply stability), at least one kind selected from the group consisting of Dioscorea japonica Thunberg, Eleutherococcus senticosus Rhizome, honey, ginger root, ripe tangerine peel, unripe tangerine peel, immature orange, malt starch syrup, jujube fruit, and non-glutinous rice is preferable; and from the viewpoint of the ease of processing for commercialization, at least one kind selected from the group consisting of Dioscorea japonica Thunberg, Eleutherococcus senticosus Rhizome, honey, ripe tangerine peel, and unripe tangerine peel is particularly preferable. The component (4) may be one or more kinds of herbal medicines, two or more kinds of herbal medicines, three or more kinds of herbal medicines, or four or more kinds of herbal medicines.

Dioscorea japonica Thunberg (Sanyaku) is a dried rhizome (rhizophore) excluding the periderm of Japanese yam (Dioscorea japonica Thunb.) or Chinese yam (Dioscorea batatas Decne.), the nature taste is flat, and sweet, the medicinal benefit coverage is spleen/lung/kidney, and the efficacy is invigorating the spleen and relieving diarrhea, nourishing yin and supporting the spleen, nourishing the lung and benefiting yin/relieving cough, and tonifying the kidney and consolidating the essence/reducing urination/arresting leucorrhea. The main components are polysaccharides (starch, and glycoprotein).

Eleutherococcus senticosus Rhizome (Shigoka) is a dried root or rhizome of Siberian ginseng (Eleutherococcus senticosus (Rupr. et Maxim.) Maxim.), the nature taste is warm, and hot/bitter, the medicinal benefit coverage is liver/kidney, and the efficacy is relieving rheumatism, replenishing the liver and kidney/strengthening the muscles and bones, and inducing diuresis and removing edema. The main components are saponin, and lignan.

Honey (Houmitsu) is a sweet substance collected from a hive of European honeybees (Apis mellifera L.) or Oriental honeybees (Apis cerana F.), the nature taste is flat, and sweet, the medicinal benefit coverage is lung/spleen/large intestine, and the efficacy is clearing the intestine for curing constipation, clearing heat, moistening the lung and relieving cough, invigorating the spleen-stomach, and relieving pain according to the urgency. The main components are fructose, and glucose.

Oriental bush cherry (Ikurinin) is a dried ripe seed of Koniwazakura (Prunus humilis Bge.), Niwaume (Prunus japonica Thunb.), or Prunus pedunculata (Pall.) Maxim., the nature taste is flat, and hot/bitter/sweet, the medicinal benefit coverage is large intestine/small intestine/spleen, and the efficacy is clearing the intestine for curing constipation, and lowering qi for moving water. The main components are a cyanogenetic glycoside, fatty oil, and flavonoid.

Ginger root (Shoukyou) is a rhizome and sometimes a dried rhizome excluding the periderm of ginger (Zingiber officinale Roscoe), the nature taste is slight warm, and hot, the medicinal benefit coverage is lung/spleen/stomach, and the efficacy is dispelling cold and resolving the superficies, warming the stomach and relieving vomiting, dissipating phlegm for moving water, and removing toxicity. The main components are essential oil, a hot taste component, and starch.

Ripe tangerine peel (Chinpi) is a dried ripe pericarp of Satsuma mandarin (Citrus unshiu (Swingle) Marcow.) or Ponkan mandarin (Citrus reticulata Blanco), the nature taste is warm, and hot/bitter, the medicinal benefit coverage is spleen/lung, and the efficacy is regulating qi-flowing for invigorating the spleen, and drying dampness and dissipating phlegm. The main components are essential oil, and flavonoid.

Unripe tangerine peel (Seihi) is a dried unripe pericarp or unripe fruit of Satsuma mandarin (Citrus unshiu (Swingle) Marcow.) or ponkan orange (Citrus reticulata Blanco), the nature taste is warm, and hot/bitter, the medicinal benefit coverage is liver/bile/spleen/stomach, and the efficacy is dispersing stagnated liver qi and breaking qi-flowing, and eliminating accumulation and stagnation. The main components are essential oil, and flavonoid glycosides.

Immature orange (Kijitsu) is a dried unripe fruit of bitter orange (Citrus aurantium L. var. daidai Makino), Citrus aurantium L., or Japanese summer orange (Citrus natsudaidai (Yu. Tanaka) Hayata), the nature taste is slight warm, and sweet, the medicinal benefit coverage is spleen/stomach/large intestine, and the efficacy is discarding and eliminating accumulation, and dissipating phlegm and dispersing glomus. The main components are essential oil, and flavonoid glycosides.

Malt starch syrup (Koui) is made by hydrolyzing and saccharifying starch of maize (Zea mays L.), cassava (Manihot esculenta Crantz), potato (Solanum tuberosum L.), sweet potato (Ipomoea batatas (L.) Poir.), or rice plant (Oryza sativa L.), or seeds excluding the seed coat of rice plant, the nature taste is slight warm, and sweet, the medicinal benefit coverage is spleen/stomach/lung, and the efficacy is replenishing deficiency and invigorating the spleen-stomach/relieving pain according to the urgency, and moistening the lung and relieving cough. The main component is maltose.

Jujube fruit (Taisou) is a dried fruit of jujube (Zizyphus jujuba Mill. var. inermis (Bunge) Rehder), the nature taste is slight warm, and sweet, the medicinal benefit coverage is spleen/stomach/heart/liver, and the efficacy is invigorating the spleen and harmonizing the stomach, nourishing the body and soothing the nerves, and moderating properties of medicine. The main components are fructose, and glucose.

Non-glutinous rice (Koubei) is a dried ripe seed of rice plant (Oryza sativa L.), the nature taste is slight cold, and sweet, the medicinal benefit coverage is spleen/stomach, and the efficacy is helping digestion, invigorating the spleen and stimulating appetite, harmonization to build up health, and weakness in the spleen and stomach. The main component is starch.

The herbal medicines as the components (1), (2), (3), and (4), which are contained in the composition of the present embodiment may be each a herbal medicine as it is, may be each a herbal medicine finely shredded or crushed, or may be each an extract of the herbal medicine. In the present specification, the extract of a herbal medicine is preferably an extract obtained by extracting the herbal medicine with a pharmaceutically acceptable aqueous solvent such as water (including hot water), ethanol, or a mixed solvent of water and ethanol. The herbal medicine extract may be prepared by a known extraction method, or may be a general soft extract, or an extract powder/extract granules.

The mixing amounts of herbal medicines as the components (1), (2), (3), and (4) contained in the composition of the present embodiment may be each appropriately set according to various conditions of the person who ingests the composition, such as body weight, age, and symptoms, may be each an amount sufficient to exert an effect of preventing, improving, or mitigating kidney-deficiency-associated symptoms or conditions of skin, hair, and nails, and are each prescribed by the weight of herbal medicine per daily dose (in a case of an extract, the weight of herbal medicine used (herbal medicine equivalent weight)) (g/day).

As the daily dose of herbal medicine, the component (1) includes 9 to 15 g of Ligustrum fruit, 6 to 15 g of yerbadetajo herb, 9 to 15 g of Lilium bulb, 9 to 15 g of Polygonatum Rhizome, 3 to 9 g of Lycium fruit, 9 to 30 g of Sesamum indicum, and 30 to 60 g of black soybean. As the daily dose of herbal medicine, the component (2) includes 9 to 15 g of Suberect spatholobus stem, 6 to 12 g of Longan aril, 3 to 9 g of wild turmeric, 3 to 9 g of zedoary root, 6 to 15 g of Luffa vegetable sponge, 3 to 9 g of Sparganium rhizome, 3 to 9 g of safflower, 9 to 15 g of Artemisia anomala, 3 to 9 g of common fenugreek seed, 9 to 15 g of Dipsacus japonicus root, 9 to 15 g of Drynaria herb, 3 to 9 g of sea horse, 3 to 9 g of Cynomorium herb, and 9 to 15 g of Astragalus Seed. As the daily dose of herbal medicine, the component (3) includes 3 to 15 g of tea leaf, 3 to 9 g of plantago seed, 3 to 9 g of plantago herb, 9 to 30 g of coix seed, 9 to 30 g of small red-bean, 15 to 30 g of corn silk, 6 to 12 g of Lophatherum herb, 15 to 30 g of Benincasa seed, 6 to 15 g of mallow seed, 9 to 30 g of knotgrass, and 6 to 12 g of Lygodium spore. As the daily dose of herbal medicine, the component (4) includes 9 to 30 g of Dioscorea japonica Thunberg, 3 to 9 g of Eleutherococcus senticosus Rhizome, 9 to 30 g of honey, 3 to 12 g of oriental bush cherry, 3 to 9 g of ginger root, 3 to 9 g of ripe tangerine peel, 3 to 9 g of unripe tangerine peel, 3 to 9 g of immature orange, 30 to 60 g of malt starch syrup, 3 to 9 g of jujube fruit, and 5 to 8 g of non-glutinous rice. In a case where multiple herbal medicines are used as the component (1), (2), (3), or (4), the daily dose of each herbal medicine may be appropriately reduced.

The composition of the present embodiment is a composition for use in preventing, improving, or mitigating kidney-deficiency-associated symptoms or conditions of skin, hair, and nails. The composition of the present embodiment supplements the blood by allowing the component (1) to act on the spleen/heart, allows the component (2) to help the circulation of the supplemented blood and life force, allows the component (3) to help the excretion of excess water that has stagnated in the body, and eliminates the kidney deficiency as a result. Further, the component (4) is expected to act on the kidney/spleen and contribute to the excretion of toxins/waste products in the body, and by acting synergistically with components (1) to (3), the component (4) achieves the exertion of an action particularly on the symptoms of skin, hair, and nails that are related to so-called beauty. As described above, by containing the components (1), (2), (3), and (4), kidney-deficiency-associated symptoms or conditions of skin, hair, and nails can be prevented, improved, or mitigated.

Kidney-deficiency-associated symptoms or conditions of skin, hair, and nails are at least one, at least two, at least three, at least four, or at least five selected from the group consisting of loose facial skin, fine wrinkles, gray hair, hair loss, dryness/lack of firmness in hair, brittle/easily-broken nails, vertical lines on nails/poor nail color, and dryness of skin, and in particular, are preferably at least one, at least two, at least three, or at least four selected from the group consisting of hair loss, dryness/lack of firmness in hair, brittle/easily-broken nails, and dryness of skin. In addition, at least one of the symptoms may be temporary, or may be a chronic symptom that continues over a period of one or more weeks, two or more weeks, four or more weeks, three or more months, six or more months, or one or more years.

The composition of the present embodiment is preferably a food composition or a pharmaceutical composition. The herbal medicines to be contained in the composition of the present embodiment are not recorded in herbal medicines and the like of The Pharmacopoeia of Japan and not regulated for use, and thus, can be used for both medical care and foods. Accordingly, the composition of the present embodiment can be a food composition such as a functional food. Therefore, there is an advantage that discomfort symptoms and physical disorders can be easily prevented, improved, or mitigated without requiring any prescription by a doctor.

The food composition may be in any possible form as food. Examples of the form include: a milk product; a fermented food (yogurt, cheese, et al.); beverages (coffee, juice, a soft drink such as a tea beverage, a carbonated beverage, a milk beverage, a lactic acid bacteria beverage, a beverage containing lactic acid bacteria, a yogurt drink, and liquors such as Japanese rice wine, fruit wine, and Western liquor); spreads (custard cream, et al.); pastes (fruit paste, et al.); Western confectionery (donuts, pies, cream puffs, chewing gum, candy, jelly, cookies, cake, chocolate, pudding, et al.); Japanese confectionery (Daifuku (soft round rice cake stuffed with sweet bean jam), rice cake, sweet bun (bun with a bean-jam filling), sponge cake, Anmitsu (agar jelly served with red bean paste and brown sugar syrup), Yokan (sweet bean jelly), et al.); ice confectionery (ice cream, popsicles, sherbet, et al.); foods (curry, Gyudon (a bowl of rice topped with boiled beef and onions), porridge of rice and vegetables, rice gruel, miso (fermented soybean paste) soup, soup, meat sauce, pasta, Japanese pickled vegetables, jam, etc.); and condiments (dressing, flavor enhancer, Furikake (dried food sprinkling over rice), broth, et al.). Such a food composition can be served as a dish at a restaurant, and can also be provided in a form of general distribution by taking a form of prepared food, retort pouch food, a form of frozen food, another packaging form or the like of a general food composition.

In addition to the components (1), (2), (3), and (4) as the active components, the food compositions in the above various forms may contain a component that is acceptable as food, as long as it does not affect the action of the active components. Examples of the component include various general foodstuffs, starch, gelatin, vegetable fat and oil, animal fat and oil, processed fat and oil or the like, carboxymethyl cellulose, gum arabic, locust bean gum, gum tragacanth, karaya gum, konjac glucomannan, xanthane gum, agar, carrageenan, polyacrylic acid, carob bean gum, alginic acid, a gellant such as a cyclodextrin, a seasoning, a sweetener, a spice, a colorant, a preservative, an antioxidant, water, an algefacient, a binder, a sweetener, a disintegrant, a lubricant, a colorant, flavor, a stabilizer, an antiseptic, a sustained release regulator, a surfactant, a solubilizer, and a wetting agent.

The food composition of the present embodiment may appropriately arbitrarily mix a functional food material for the purpose of further exerting a complex effect. As the functional food material, for example, various kinds of herbal medicines other than the components (1), (2), (3), and (4), various kinds of probiotics, various kinds of oligosaccharides, nutrient components such as vitamins and minerals, flavonoids, quinones, polyphenols, amino acid, nucleic acid, essential fatty acid, and other known functional food materials may be appropriately arbitrarily mixed.

The production method of food compositions in various forms is not particularly limited, and a known method can be used appropriately. Further, a component capable of reducing the peculiar taste and smell of a herbal medicine may be added, or treatment with such a component may be performed.

The food composition of the present embodiment may also be a food for the purpose of preservation of health, health maintenance, health promotion, and the like, for example, a health food, a food with function claims, a nutritional supplementary food, a supplement, a food for specified health uses, a food with nutrient function claims, or the like. In a case of being a supplement, the food composition of the present embodiment may be in a form of, for example, tablets, capsules, granules, a liquid, a powder, a drink, jelly, or the like. The food composition of the present embodiment may be ingested as it is, or may be mixed into meals, snacks, beverages, or the like.

The contents of components (1), (2), (3), and (4) as active components in the food composition of the present embodiment are not particularly limited, and vary depending on the form of the food composition. For example, in a case where the food composition is in a form of tablets/capsules/granules/a liquid/a powder/a supplement such as stick jelly, the total weight of each herbal medicine in the food composition can be mentioned to be typically more than 0 to 100% by weight, for example, 10% to 80% by mass of the food composition. Further, in a case where the form of the food composition is a form of various kinds of beverages/jelly, or a form of general foods such as foods/retort pouch foods, the total weight of each herbal medicine in the food composition can be mentioned to be typically 0.05 to 25% by mass of the food composition. Considering the taste and the amount, it is preferable to minimize the mixing amounts of the active components in a food composition, and in that case, it is preferable to use an extract of a herbal medicine rather than mixing the herbal medicine as it is. Since the herbal medicine contained in the food composition of the present embodiment has usually a mild effect when used as a food, the intake is not particularly limited. If the intake of the food composition of the present embodiment is an amount that includes the daily doses of the above-described herbal medicines, the desired effects of preventing, improving, or mitigating symptoms relating to qi deficiency and/or blood deficiency can be obtained.

The pharmaceutical composition of the present embodiment may be in a form of a drink, syrup, jelly, a lyophilized powder, tablets, capsules, pills, a powder, fine granules, granules, or the like, and may contain in addition to the components (1), (2), (3), and (4) as the active components, for example, a pharmaceutically acceptable component, for example, an excipient, a binder, a lubricant, a disintegrant, an emulsifier, a surfactant, a base, a dissolution aid, or a suspending agent.

The composition of the present embodiment may also be used in combination with other compositions. For example, by using the composition of the present embodiment in combination with an over-the-counter drug that is used to treat symptoms relating to qi deficiency and/or blood deficiency, for example, a vitamin B, a liver hydrolysate, or the like, discomfort symptoms can be eliminated with minimal use of the over-the-counter drug.

The composition may be ingested once a day, or may be ingested in several divided doses, for example, 2, 3 or 4 divided doses. In a case of ingesting the composition in several divided doses, the composition should be ingested so that the total doses are the daily dose. The timing of the ingestion of the composition of the present embodiment is not particularly limited, and in a case where the ingestion is once a day, the composition may be taken at any time such as after waking up, before meal, between meals, after meal, or before going to bed, and in a case where the ingestion is several times a day, the composition may be taken at two, three, or four or more of timings of after waking up, before meal, between meals, after meal, and before going to bed. Further, the composition may be ingested together with meal.

The composition of the present embodiment may be ingested continuously over a period of one or more weeks, two or more weeks, four or more weeks, three or more months, six or more months, or one or more years. Since the composition of the present embodiment is a safe herbal medicine component, it is safe to ingest the composition over a long period of time, and by taking the composition for a long period of time, the physical constitution is gradually improved, and kidney-deficiency-associated symptoms or conditions of skin, hair, and nails can be gradually mitigated or improved.

The method of one embodiment is a method for preventing, improving, or mitigating kidney-deficiency-associated symptoms or conditions of skin, hair, and nails, including administering the composition of the above present embodiment to a subject in need thereof. In this regard, the subject is a subject who is aware of kidney-deficiency-associated symptoms or conditions of skin, hair, and nails, or has been diagnosed with kidney deficiency based on Chinese medicine, and has kidney-deficiency-associated symptoms or conditions of skin, hair, and nails. The composition is given (for example, administered) to the subject in the daily dose described above. The administration period is not particularly limited, and may be one or more weeks, two or more weeks, four or more weeks, three or more months, six or more months, or one or more years.

The composition of one embodiment is a composition for use in preventing, improving, or mitigating kidney-deficiency-associated symptoms or conditions of skin, hair, and nails, and the composition may be a composition of the above present embodiment. In this regard, the use method (intake, and intake period) is as described above.

The use of one embodiment is use of the composition of the present embodiment, in producing a composition for preventing, improving, or mitigating kidney-deficiency-associated symptoms or conditions of skin, hair, and nails. Further, the use of another embodiment is use of the composition of the present embodiment, in preventing, improving, or mitigating kidney-deficiency-associated symptoms or conditions of skin, hair, and nails. In this regard, the use method (intake, and intake period) is as described above. The composition may be a food composition or a pharmaceutical composition, and the composition can be produced according to the usual method so as to include an effective amount of herbal medicine or herbal medicine extract of the components (1), (2), (3), and (4), and may include other components described above if necessary.

Hereinafter, examples will be given in order to describe the present invention further in detail. However, the present invention should not be limited at all to these examples.

### EXAMPLES

### Test Example 1

### (Evaluation Test of Effect on Kidney-Deficiency-Associated Physical Disorder)

A single-blind controlled before-after trial was conducted on 18 individuals (Example 1), 16 individuals (Example 2 and Comparative Example 1), 14 individuals (Example 3), 12 individuals (Example 4), or 15 individuals (Example 5), who were Japanese men and women aged 20 or more and had been aware of daily physical disorders caused by kidney deficiency, and the effects of the present invention were investigated.

### (Composition)

The composition of herbal medicines and the amounts used for daily dose of Examples 1, 2, 3, 4, and 5, and Comparative Example 1 are shown in Table 1. As the raw material herbal medicines, herbal medicines were mixed according to the ratio of herbal medicines listed in Table 1, the mixture was boiled and extracted in water, the filtration, concentration, and sterilization were performed, dextrin in an adequate amount was added to the obtained product, and then the resultant product was dried with a spray dryer to obtain a mixed plant extract powder. In the obtained extracts, as the raw material herbal medicine, 6.0 g of extract powder of Example 1, 13.8 g of extract powder of Example 2, 14.4 g of extract powder of Example 3, 15.6 g of extract powder of Example 4, 11.4 g of extract powder of Example 5, or 6.0 g of extract powder of Comparative Example 1, equivalent to the amount of herbal medicines listed in Table 1 was used as a food composition for one day.

TABLE 1

| | Component (1)/g | Component (2)/g | Component (3)/g | Component (4)/g |
|---|---|---|---|---|
| Example 1 | Ligustrum fruit/9 | Suberect spatholobus | Tea leaf/3 | Dioscorea japonica |
| | Yerbadetajo herb/3 | stem/9 | | Thunberg/4.5 |
| | | | | Eleutherococcus |
| | | | | Senticosus Rhizome/3 |
| Example 2 | Ligustrum fruit/9 | Wild turmeric/3 | Small red-bean/9 | Honey/9 |
| | | Suberect spatholobus | | |
| | | stem/9 | | |
| Example 3 | Lycium fruit/6 | Zedoary root/3 | Coix seed/9 | Ripe tangerine peel/4.5 |
| | | Longan aril/3 | Lophatherum herb/6 | Non-glutinous rice/5 |
| Example 4 | Lilium bulb/9 | Safflower/3 | Plantago seed/3 | Jujube fruit/3 |
| | Sesamum indicum/9 | Luffa vegetable | | Malt starch syrup/3 |
| | | sponge/6 | | |
| Example 5 | Yerbadetajo herb/3 | Common fenugreek | Plantago herb/3 | Unripe tangerine |
| | Black soybean/20 | seed/3 | Corn silk/15 | peel/4.5 |
| | | | | Immature orange/3 |
| | | | | Ginger root/3 |
| Comparative | Ligustrum fruit/9 | — | Tea leaf/3 | Dioscorea japonica |
| Example 1 | | | | Thunberg/4.5 |
| | | | | Eleutherococcus |
| | | | | Senticosus Rhizome/3 |

Each of subjects ingested one dose of the extract powder of Example 1, 2, 3, 4 and 5 or Comparative Example 1 (Example 1:2.0 g, Example 2:4.6 g, Example 3:4.8 g, Example 4:5.2 g, Example 5:3.8 g, and Comparative Example 1:2.0 g) continuously for two weeks (14 days) once a day before breakfast after dissolving the powder in about 150 cc of warm water. Discomfort questionnaires were conducted for all the subjects before the ingestion and at the completion of the ingestion for 2 weeks, and the degree of improvement in the symptoms was evaluated. In the discomfort questionnaires before the ingestion, for each symptom group listed in Table 2, scores of 3 levels: “extremely worried (1)”, “slightly worried (2)”, and “not worried (3)” were set, and the presence or absence of awareness of each symptom was evaluated. In the discomfort questionnaires at the completion of the ingestion, for each symptom group listed in Table 2, scores of 5 levels: “very improved (1)”, “improved (2)”, “unchanged (3)”, “impaired (4)”, and “very impaired (5)” were set, and the presence or absence of improvement of each symptom was evaluated. The proportion of the subjects of which each score of the questionnaire at the completion of the ingestion was 1 or 2 among the subjects of which each score of the questionnaire before the ingestion was 1 or 2 was taken as the improvement rate, and the extract with an improvement rate of a positive value was determined to have an effect on the improvement of the discomfort symptom, and the extract with an improvement rate of 30% or more was determined to have a remarkable effect on the improvement of the discomfort symptom.

### Results

The results are shown in Table 2. From Table 2, it was observed that hair loss, dryness/lack of firmness in hair, brittle/easily-broken nails, and dryness of skin were improved in subjects who ingested the herbal medicine extracts of Examples 1, 2, 3, 4 and 5 containing the components (1), (2), (3), and (4) as compared with the subjects who ingested the herbal medicine extract of Comparative Example 1 containing the components (1), (3), and (4). In addition to the effects, it was observed that chronic fatigue/shortness of breath, heavy/sluggish in the body, and insomnia/sleep disorder were improved by the ingestion of the herbal medicine extracts of Examples 1, 2, 3, 4 and 5. Further, it was observed that loose facial skin, fine wrinkles, gray hair, and vertical lines on nails/poor nail color were improved by the ingestion of the herbal medicine extract of Example 1; loose facial skin, and vertical lines on nails/poor nail color were improved by the ingestion of the herbal medicine extract of Example 2; gray hair, and vertical lines on nails/poor nail color were improved by the ingestion of the herbal medicine extract of Example 3; loose facial skin, fine wrinkles, and gray hair were improved by the ingestion of the herbal medicine extract of Example 4; and gray hair, and vertical lines on nails/poor nail color were improved by the ingestion of the herbal medicine extract of Example 5. That is, the effects on the prevention, improvement, or mitigation were confirmed in the whole symptoms relating to kidney deficiency based on the synergistic action of the components (1), (2), (3), and (4).

TABLE 2

| | | | | | | | | | | | | Difficulty |
|---|---|---|---|---|---|---|---|---|---|---|---|---|
| | | | Concerned | | | | | Vertical | | | | in falling |
| | | | about fine | | | | | lines on | | Chronically | | asleep/ |
| | | Feeling | wrinkles on | Concerned | Excessive | Dryness/ | Brittle/ | nails/ | Dry- | easy to | Heavy/ | poor sleep |
| | | of loose | forehead | about | hair loss/ | lack of | easily- | poor | ness | get tired/ | sluggish | (insomnia/ |
| | | facial | and around | gray | increased | firmness | broken | nail | of | shortness | in the | sleep |
| Group | Item | skin | eyes | hair | hair loss | in hair | nails | color | skin | of breath | body | disorder) |
| Example 1 | Improvement | 20 | 14 | 8 | 36 | 20 | 13 | 29 | 6 | 17 | 7 | 33 |
| | rate (%) | | | | | | | | | | | |
| | Improvement | ◯ | ◯ | ◯ | ⊙ | ◯ | ◯ | ◯ | ◯ | ◯ | ◯ | ⊙ |
| Example 2 | Improvement | 10 | 0 | 0 | 33 | 38 | 14 | 9 | 17 | 17 | 13 | 43 |
| | rate (%) | | | | | | | | | | | |
| | Improvement | ◯ | | | ⊙ | ⊙ | ◯ | ◯ | ◯ | ◯ | ◯ | ⊙ |
| Example 3 | Improvement | 0 | 0 | 17 | 44 | 33 | 20 | 33 | 30 | 50 | 33 | 33 |
| | rate (%) | | | | | | | | | | | |
| | Improvement | | | ◯ | ⊙ | ⊙ | ◯ | ⊙ | ⊙ | ⊙ | ⊙ | ⊙ |
| Example 4 | Improvement | 25 | 17 | 20 | 63 | 57 | 17 | 0 | 10 | 40 | 50 | 50 |
| | rate (%) | | | | | | | | | | | |
| | Improvement | ◯ | ◯ | ◯ | ⊙ | ⊙ | ◯ | | ◯ | ⊙ | ⊙ | ⊙ |
| Example 5 | Improvement | 0 | 0 | 14 | 33 | 50 | 33 | 22 | 18 | 57 | 25 | 25 |
| | rate (%) | | | | | | | | | | | |
| | Improvement | | | ◯ | ⊙ | ⊙ | ⊙ | ◯ | ◯ | ⊙ | ◯ | ◯ |
| Comparative | Improvement | 0 | 0 | 0 | 0 | 14 | 0 | 0 | 10 | 14 | 9 | 25 |
| Example 1 | rate (%) | | | | | | | | | | | |
| | Improvement | | | | | ◯ | | | ◯ | ◯ | ◯ | ◯ |

⊙: There is a remarkable effect on the improvement.
◯: There is an effect on the improvement.

1. A composition, comprising as active components the following components (1), (2), (3), and (4):
component (1): at least one kind of herbal medicine selected from the group consisting of Ligustrum fruit, yerbadetajo herb, Lilium bulb, Polygonatum Rhizome, Lycium fruit, Sesamum indicum, and black soybean;
component (2): at least one kind of herbal medicine selected from the group consisting of Suberect spatholobus stem, Longan aril, wild turmeric, zedoary root, Luffa vegetable sponge, Sparganium rhizome, safflower, Artemisia anomala, common fenugreek seed, Dipsacus japonicus root, Drynaria herb, sea horse, Cynomorium herb, and Astragalus Seed;
component (3): at least one kind of herbal medicine selected from the group consisting of tea leaf, plantago seed, plantago herb, coix seed, small red-bean, corn silk, Lophatherum herb, Benincasa seed, mallow seed, knotgrass, and Lygodium spore; and
component (4): at least one kind of herbal medicine selected from the group consisting of Dioscorea japonica Thunberg, Eleutherococcus senticosus Rhizome, honey, oriental bush cherry, ginger root, ripe tangerine peel, unripe tangerine peel, immature orange, malt starch syrup, jujube fruit, and non-glutinous rice.

2. The composition according to claim 1, wherein the composition is for use in preventing, improving, or mitigating kidney-deficiency-associated symptoms or conditions of skin, hair, and nails.

3. The composition according to claim 2, wherein the kidney-deficiency-associated symptoms or conditions of skin, hair, and nails is at least one selected from the group consisting of loose facial skin, fine wrinkles, gray hair, hair loss, dryness/lack of firmness in hair, brittle/easily-broken nails, vertical lines on nails/poor nail color, and dryness of skin.

4. The composition according to claim 1, wherein the composition is a food composition or a pharmaceutical composition.
